(19) United States
(12) Patent Application Publication
(10) Pub. No.: US 2019/0366331 A1
(43) Pub. Date: Dec. 5, 2019

(54) Assay plate and manufacturing method thereof

(71) Applicant: Tunghai University, Taichung (TW)

(72) Inventor: Feng-Di LUNG, Taichung (TW)

(21) Appl. No.: 15/380,228

(22) Filed: Dec. 15, 2016

(51) Int. Cl. B01L 3/00; C07D 207/40

(52) U.S. Cl. CPC B01L 3/5085; C07D 207/40; B01L 3/5025

(57) ABSTRACT

The present invention discloses an assay plate, which has a plate body made of polymeric material modified by coating a compound A thereon, and allows a molecule such as protein or peptide, or a group to bind to the plate body by hydrophobic bonding for use in biomedical assay.

### TECHNICAL FIELD

The present invention relates to an assay tool, and particularly to an assay plate and manufacturing method thereof.

### PRIOR ART

Conventional enzyme-linked immunosorbent assays (ELISAs) are widely used in immunodiagnosis of various diseases by utilizing the specificity between an antigen and an antibody to detect a marker. The ELISA assay plate is mostly made of polyvinyl chloride or polystyrene to be a mixture with high hydrophobicity.

Generally speaking, the marker may be protein, peptide, epitope, bacteria, virus, sugar, lipid or deoxyribonucleic acid, wherein t, the protein, peptide, or other marker with polarity has no desirable binding force with the ELISA assay plate of hydrophobic material, so that the marker can't be directly bound to the ELISA assay plate. In order to enhance and improve the binding force of the ELISA assay plate to the marker, it has to use an acidic coating buffer to enhance the hydrophobic bonding. Therefore, in the prior art, there is still a need for techniques that can effectively improve the binding force between the ELISA assay plate and the marker.

### SUMMARY OF THE INVENTION

Therefore, a main object of the present invention is to provide an assay plate and a manufacturing method thereof, which can allow a biomarker such as peptide or protein to bind to the assay plate, so as to effectively improve the bonding rate of the biomarker on the assay plate.

To achieve the above object, the assay plate provided can be used in immunology, serology, epidemiology, and assay of diseases such as agricultural diseases, by virtue of the specific reaction between the antigens and antibodies. The assay plate mainly comprises a plate body and a plurality of compounds A, wherein:

the plate body, may made of polystyrene, a polyvinyl chloride derivative or other polymeric materials with high hydrophobicity, comprises a body, and at least one cavity c of an predetermined internal diameter located in the body and formed by extending downwards to an predetermined depth from an end face on one side of the body.

Each of the compounds A is disposed in the cavity, and has a structure represented by Formula I:

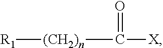

wherein:
R₁ is a hydrophobic group, for example, phenyl or other hydrophobic groups; —(CH₂)ₙ— is a hydrocarbon group composed of carbon and hydrogen atoms; and
X is —OH,

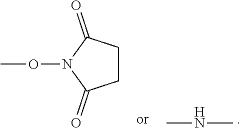

In embodiments of the present invention, each of the compounds A binds to the plate body through a hydrophobic force between R₁ and the plate body; —(CH₂)ₙ— is linear, and 1≤n≤3, so as to avoid overlapping of the carbon chains with each other which affects the effect of coating of the compounds A onto the plate body.

For example, each of the compound A is phenylacetic acid, a succinate or an amide derivative.

Also, each of the compound A uses

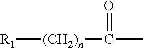

as a linker between X and the plate body, wherein the R₁ end of the linker is used for having the hydrophobic force with the plate body, and the other end of the linker t is to provide a carbonyl group for binding to X.

Furthermore, the present invention discloses a method for manufacturing the assay plate, including binding at least one compound A to a plate body by compound A's hydrophobic end, wherein the compound A is selected from the group consisting of carboxylic acid, succinate compound or amide compound.

In the embodiments of the present invention, the method for manufacturing the assay plate comprises the following steps:

Step a: contacting at least one compound A with the plate body, to allow a moiety of the compound A to bind to a surface of the plate body.

Step b: obtaining an assay plate.

Preferably, in Step a, the compound A is contacted with the plate body for at least 6 hours.

Preferably, in Step a, the compound A is contacted with the plate body by coating, perfusing or soaking.

Preferably, after Step a, a dry procedure is carried out, to remove materials that do not bind to the plate body.

In embodiments of the present invention, the compound A in Step a is phenylacetic acid, and the manufacturing method further comprises a Step a1 between Step a and Step b, wherein:

Step a1: providing an activating reagent to react with the compound A bound to the plate body, wherein the activating reagent comprises EDC (N′-(Ethylcarbonimidoyl)-N,N-dimethylpropane-1,3-diaminemonohydrochloride) and NHS (N-oxysuccinimide).

Preferably, EDC is mixed with NHS at a molar ratio of 5:1.

Preferably, the reaction time of the activating reagent with the compound A is at least 10 mins.

Furthermore, to enable the assay plate of the present invention to be used for detecting a target analyte, in the embodiments of the present invention, the manufacturing method further includes Steps a2 and a3, sequentially between Steps a1 and b, wherein:

Step a2: providing a marker to react with the succinate compound obtained in Step a1, to obtain an amide compound, wherein the marker is an amine or ammonia derivative; and

Step a3: masking the un-reacted succinate compound in Step a2 with ethanolamine.

Preferably, in Step a2, the reaction time of the marker with the succinate compound is at least 1 hour.

Preferably, in Step a3, the masking reaction is continued for at least 1 hour.

Preferably, the reaction in Step a2 occurs in an acidic environment. For example, the reaction is carried out in an environment containing sodium acetate, or in an environment with pH 3-5.

### BRIEF DESCRIPTION OF ACCOMPANYING DRAWINGS

FIG. 1 is a stereogram of a first embodiment of the present invention.

FIG. 2 is a schematic stereogram of a part of the plate body in a fourth embodiment of the present invention, in which phenylacetic acid is attached to a surface of the plate body through hydrophobic binding.

FIG. 3 is a schematic stereogram of a part of the plate body in the fourth embodiment of the present invention, in which the carboxyl group of phenylacetic acid is activated with EDC and NHS.

FIG. 4 is a schematic stereogram of a part of the plate body in the fourth embodiment of the present invention, in which a marker is indirectly immobilized, through a linker.

FIG. 5 is a schematic stereogram of a part of the plate body in the fourth embodiment of the present invention, in which a succinate that is not bound to the marker is masked by ethanolamine.

FIG. 6 shows a standard curve plotted by indirect ELISA using a conventional ELISA plate, with the maximum concentration being 80 mg/mL.

FIG. 7 shows a standard curve plotted by indirect ELISA using a conventional ELISA plate, with the maximum concentration being 40 mg/mL.

FIG. 8 shows a standard curve plotted by indirect ELISA using a conventional ELISA plate, with the maximum concentration being 32 mg/mL.

FIG. 9 shows a standard curve plotted by indirect ELISA using a conventional ELISA plate, with the maximum concentration being 16 mg/mL.

FIG. 10 shows a standard curve plotted by indirect ELISA using an assay plate fabricated using the method disclosed herein, with the maximum concentration being 2 mg/mL.

FIG. 11 shows a standard curve plotted by indirect ELISA using an assay plate fabricated using the method disclosed herein, with the maximum concentration being 250 μg/mL.

### DETAILED DESCRIPTION

The present invention discloses an assay plate, which has a plate body made of a polymeric material modified by coating at least one compound A thereon, and allows a molecule such as protein or peptide or a chemical group to bind to the plate body by hydrophobic bonding, for use in biomedical assay. The compound A has a structure represented by Formula I:

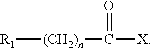

Furthermore, the compound A comprises a linker:

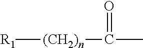

and X, wherein the linker is used to link X to the plate body. In embodiments of the present invention, R₁ in the compound A is a hydrophobic group, for example, phenyl; the —(CH₂)ₙ— group is linear and 1≤n≤3; and X is —OH,

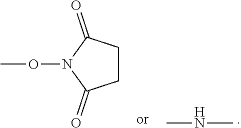

It thus can be known that, in order to provide various uses, X is bound to —(CH₂)ₙ— via a carbonyl group, and by changing or selecting the structure or type of X, the assay plate can be modified with different compounds.

Therefore, the assay plate as disclosed in the present invention is formed based on the fact that the plate body has thereon a coating containing a phenyl ring or carbon chain, and a hydrophobic force or Van Der Waals force is formed between the compound A and the plate body through the hydrophobic group R₁ that is to be attached to the plate body.

For example, when X is carboxyl, the assay plate is modified by a compound A that is a carboxylic acid, such as phenylacetic acid.

When X is

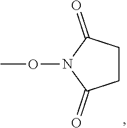

the assay plate is modified by a compound A having a structure represented by

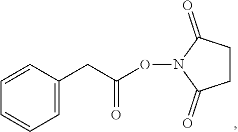 [SMILES: O=C(Cc1ccccc1)ON1C(=O)CCC1=O]

and the compound A can be formed by binding

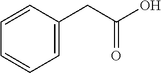

to the plate body, and then activated by a carboxyl group. Alternatively, the compound A is prepared directly and then bound to the plate body.

When X is

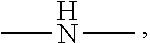

the compound A has a structure of

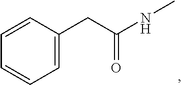

and the compound A can bind, on the N end, to a marker, to form a structure represented by

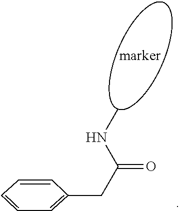

Scientific terminologies that are not noted particularly herein should be explained according to the general meaning as understood by those of ordinary skill in the art to which the present invention belongs.

The “marker” as used herein refers to a molecule whose reaction with a material to be analyzed can be measured by a biological analysis method, such as proteins, peptides, epitopes, bacteria, viruses, sugars, lipids or deoxyribonucleic acids.

The “linker” as used herein refers to one able to link the plate body disclosed herein to the marker and is formed by an organic compound. In embodiments disclosed herein, the linker has a hydrophobic end for hydrophobic bonding to the plate body, and the hydrophobic end consists of a hydrophobic group, for example, phenyl.

Further explanation will be made below in combination with several embodiments of the present invention.

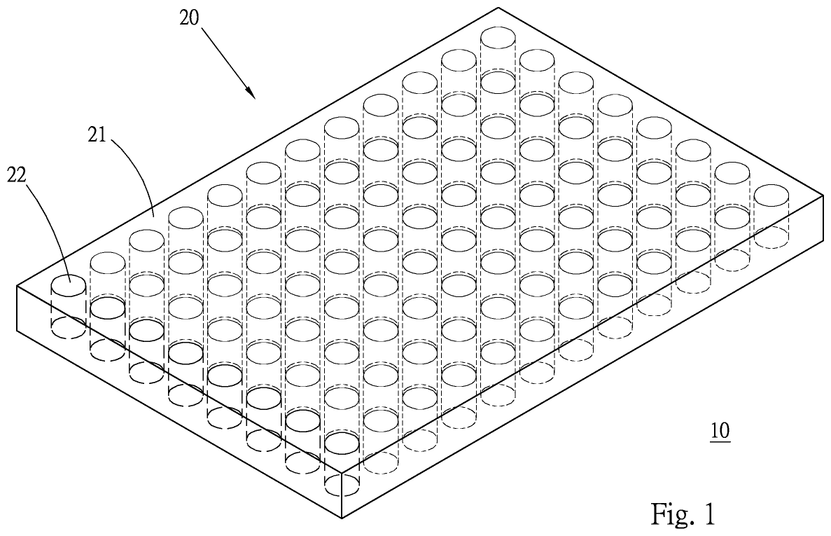

Referring to FIG. 1, an assay plate (10) provided in a first embodiment of the present invention mainly comprises a plate body (20) and a compound A.

The plate body (20) assumes a transparent plate, and is made of a material of high hydrophobicity, for example, polystyrene. The plate body (20) has a body (21), and at least one cavity (22) of an appropriate internal diameter located in the body (21) and formed by extending downwards to an appropriate depth from an end face on one side of the body (21), for accommodating the compound A.

The compound A is phenylacetic acid, with one hydrophobic end having a phenyl group and bonded to the surface of the cavity (22), such that the compound A is coated onto the surface of the cavity (22), and with the other end having un-activated carboxyl, which is to be activated for binding other molecules.

The assay plate provided in a second embodiment of the present invention has a structure substantially the same as that provided in the first preferred embodiment of the present invention, except that the compound A has a structure represented by Formula II:

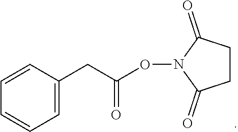 [SMILES: O=C(Cc1ccccc1)ON1C(=O)CCC1=O]

Therefore, a coated texture with a succinate compound can be formed on a surface of the cavity, which can directly bind to a molecule such as a marker through activated carboxyl in the compound.

The assay plate provided in a third embodiment of the present invention is different from that provided in the first embodiment of the present invention in that, the compound A has a structure represented by Formula III:

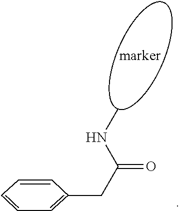

Therefore, a surface of the cavity of the assay plate may have a coated texture with an amide compound, and the other end of the compound has a marker binding to a material to be analyzed. Therefore, the assay plate can be directly used as an assay platform or an assay tool in the biomedical field.

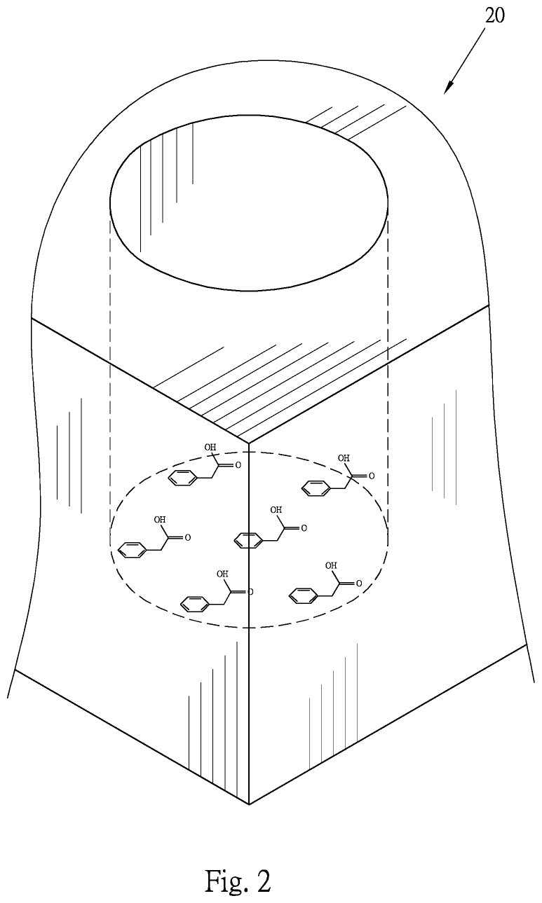

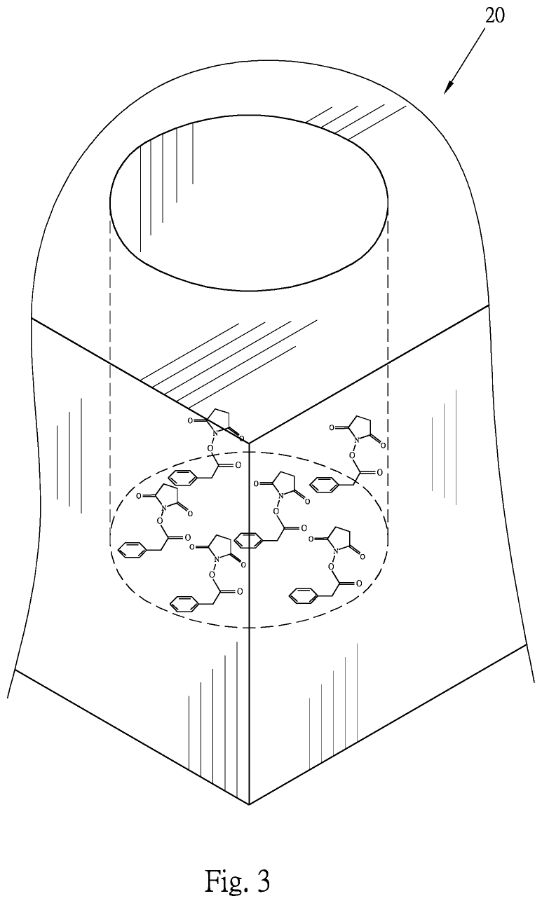

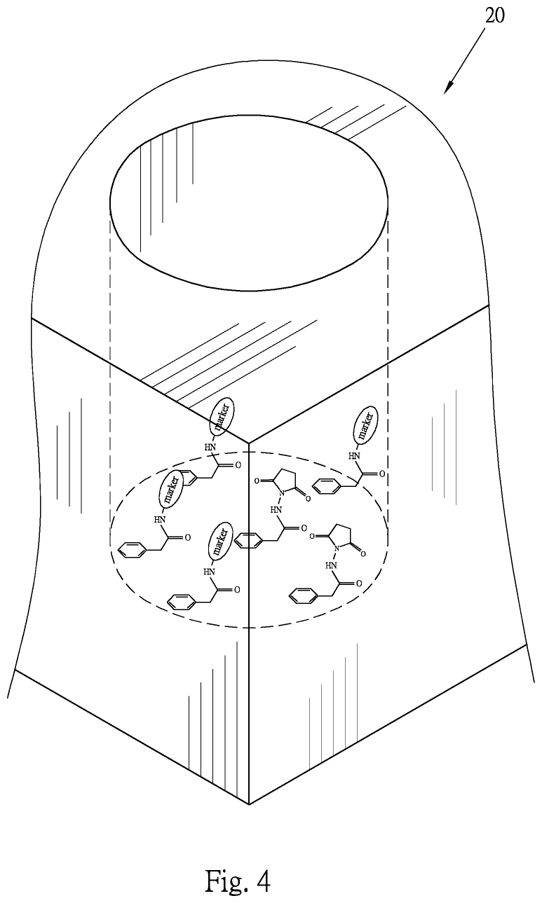

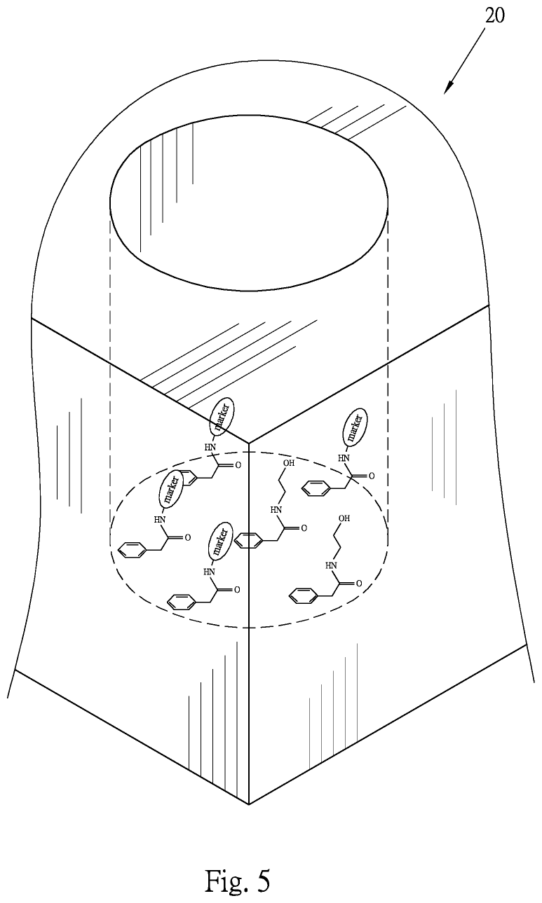

Referring to FIGS. 2 to 5, a method for manufacturing the assay plate provided in a fourth embodiment of the present invention comprises the following steps:

Step a: taking a phenylacetic acid solution and a plate body (20), binding the phenyl ring of phenylacetic acid to a phenyl ring or a carbon chain coated on the plate body, and exposing un-activated carboxyl, to obtain a plate body (20) modified with phenylacetic acid.

Step b: adding EDC and NHS to the plate body (20) modified with phenylacetic acid to activate the carboxyl group by esterification, so as to obtain a plate body (20) modified with a succinate compound, wherein the reaction formula:

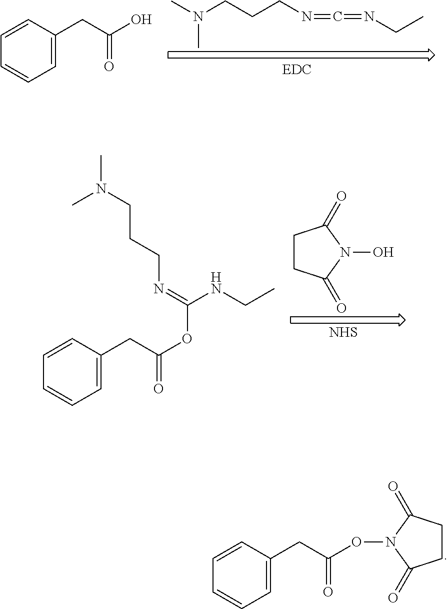

Step c: adding a sodium acetate buffer at pH 3.5 containing a peptide marker to the plate body (20) modified with the succinate compound, to carry out the aminolysis and nucleophilic substitution of the ester, such that the peptide marker is covalently bonded to the phenylacetic acid, thereby obtaining a plate body (20) modified with the peptide marker, wherein the reaction formula is:

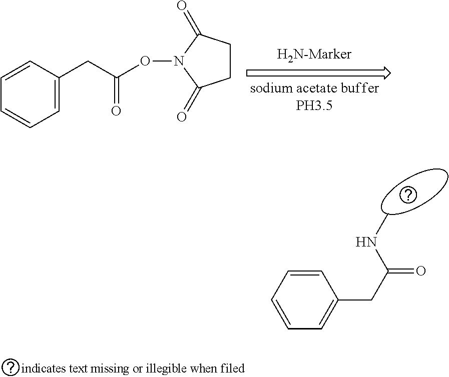 [SMILES: O=C(Cc1ccccc1)ON1C(=O)CCC1=O ; CNC(=O)Cc1ccccc1]

Step d: adding an aqueous ethanolamine solution to the plate body (20) modified with the peptide marker, to mask positions in the succinate compound that are not reacted with the peptide marker by ethanolamine, so as to obtain the assay plate disclosed herein.

To illustrate the present invention clearly, the present invention is illustrated below by way of example.

### Example 1: Preparation of Assay Plate

100 μL of a 6 mg/mL aqueous phenylacetic acid solution was added to a 96-well plate, and stood still for 6 h or more at room temperature. After the aqueous phenylacetic acid solution was removed from the plate, the plate was washed with a phosphate buffer and then dried.

Then, 100 μL of an EDC/NHS activating reagent was added to the plate, and mixed for about 10 min at room temperature, wherein the EDC/NHS activating reagent comprised 50 μL of a 0.5 M aqueous EDC solution and 50 μL of a 0.1 M aqueous NHS solution. The EDC/NHS activating reagent was removed from the plate. 100 μL a peptide/sodium acetate buffer (pH about 3.5) with a concentration of about 0.01 to 0.2 mg/mL, was added to the plate, and mixed for about 1 h at room temperature, wherein the peptide had a sequence as shown in SEQ ID NO.1. Thereafter, the peptide/sodium acetate buffer was removed from the plate, and then the plate was washed with a phosphate buffer. 200 μL of a 1 M aqueous ammonium acetate solution was added, and mixed for about 1 h at 37° C. Then the aqueous ammonium acetate solution was removed, and the plate was washed with a phosphate buffer, to obtain a peptide-modified assay plate.

### Example 2: Comparison of Assay Results

A peptide as shown in SEQ ID NO.1 was immobilized to a conventional ELISA plate by bovine serum albumin, to obtain a conventional modified ELISA plate.

The peptide-modified assay plate obtained in Example 1 and the conventional modified ELISA plate were used for assaying antibodies against the peptide as shown in SEQ ID NO.1 by indirect ELISA assay. The steps of indirect ELISA assay are ordinary knowledge in the art to which the present invention belongs, and will not be described herein.

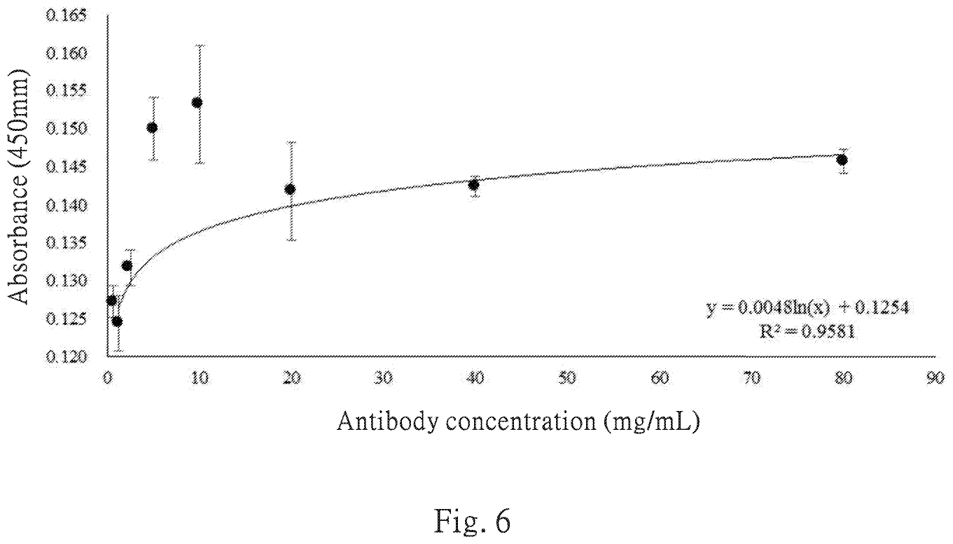

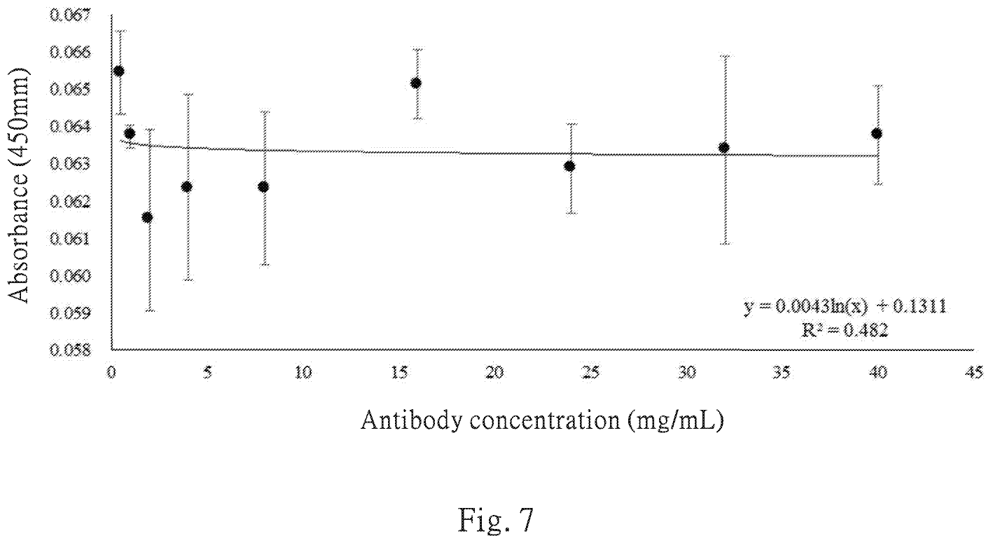

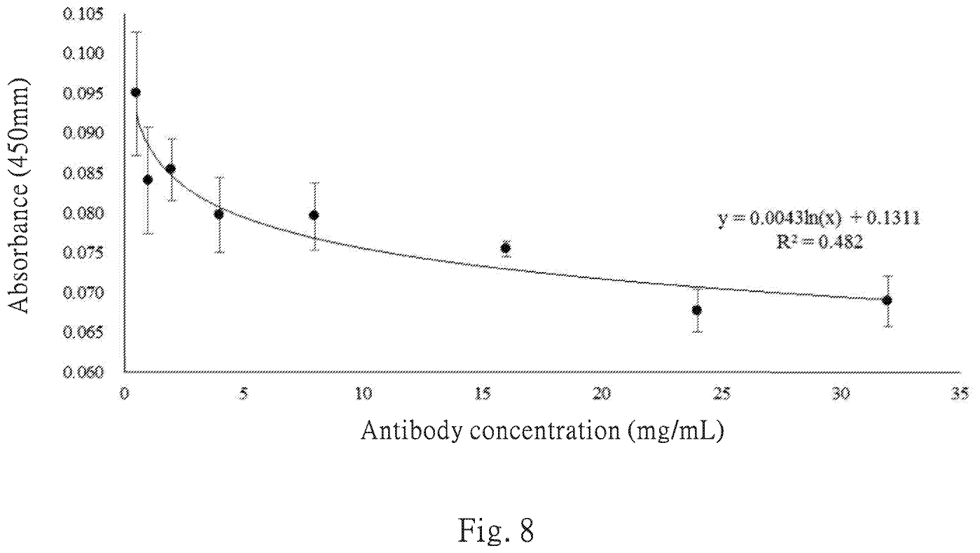

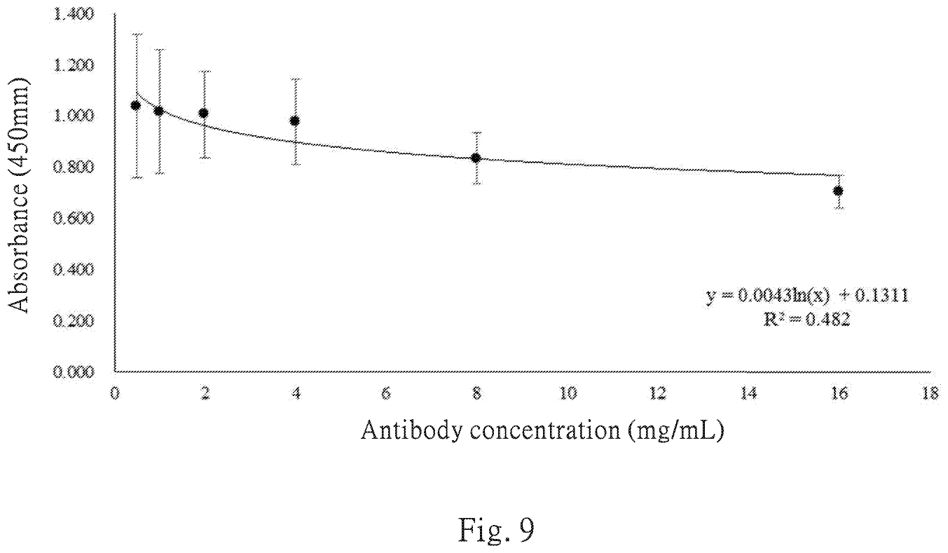

The conventional modified ELISA plate was used. The maximum antibody concentrations were 80 mg/mL, 40 mg/mL, 32 mg/mL, and 16 mg/mL respectively, and 2-fold diluted successively. The standard curves obtained are as shown in FIGS. 6 to 9, and related data is shown in Tables 1 to 4 below.

TABLE 1
Data determined at a maximum antibody concentration of 80 mg/mL

| | | | | | | | | |
|---|---|---|---|---|---|---|---|---|
| concentration | 80 | 40 | 20 | 10 | 5 | 2.5 | 1.25 | 0.625 |
| (mg/mL) | | | | | | | | |
| Average | 0.14570 | 0.14240 | 0.14177 | 0.15320 | 0.14995 | 0.13175 | 0.12437 | 0.12725 |
| Absorbance | | | | | | | | |
| Standard | 0.00158 | 0.00130 | 0.00636 | 0.00780 | 0.00405 | 0.00235 | 0.00370 | 0.00215 |
| Deviation | | | | | | | | |

TABLE 2
Data determined at a maximum antibody concentration of 40 mg/mL

| | | | | | | | | | |
|---|---|---|---|---|---|---|---|---|---|
| concentration | 40 | 32 | 24 | 16 | 8 | 4 | 2 | 1 | 0.5 |
| (mg/mL) | | | | | | | | | |
| Average | 0.06377 | 0.06337 | 0.06287 | 0.06513 | 0.06235 | 0.06237 | 0.06150 | 0.06373 | 0.06543 |
| Absorbance | | | | | | | | | |
| Standard | 0.00132 | 0.00250 | 0.00121 | 0.00092 | 0.00205 | 0.00249 | 0.00243 | 0.00031 | 0.00111 |
| Deviation | | | | | | | | | |

TABLE 3
Data determined at a maximum antibody concentration of 32 mg/mL

| | | | | | | | | |
|---|---|---|---|---|---|---|---|---|
| concentration | 32 | 24 | 16 | 8 | 4 | 2 | 1 | 0.5 |
| (mg/mL) | | | | | | | | |
| Average | 0.06895 | 0.06770 | 0.07550 | 0.07955 | 0.07973 | 0.08533 | 0.08405 | 0.09495 |
| Absorbance | | | | | | | | |
| Standard | 0.00314 | 0.00268 | 0.00099 | 0.00420 | 0.00462 | 0.00385 | 0.00663 | 0.00773 |
| Deviation | | | | | | | | |

TABLE 4
Data determined at a maximum antibody concentration of 16 mg/mL

| | | | | | | |
|---|---|---|---|---|---|---|
| concentration | 16 | 8 | 4 | 2 | 1 | 0.5 |
| (mg/mL) | | | | | | |
| Average | 0.70353 | 0.83413 | 0.97638 | 1.00578 | 1.01680 | 1.03888 |
| Absorbance | | | | | | |
| Standard | 0.06237 | 0.10113 | 0.16683 | 0.16891 | 0.24067 | 0.28001 |
| Deviation | | | | | | |

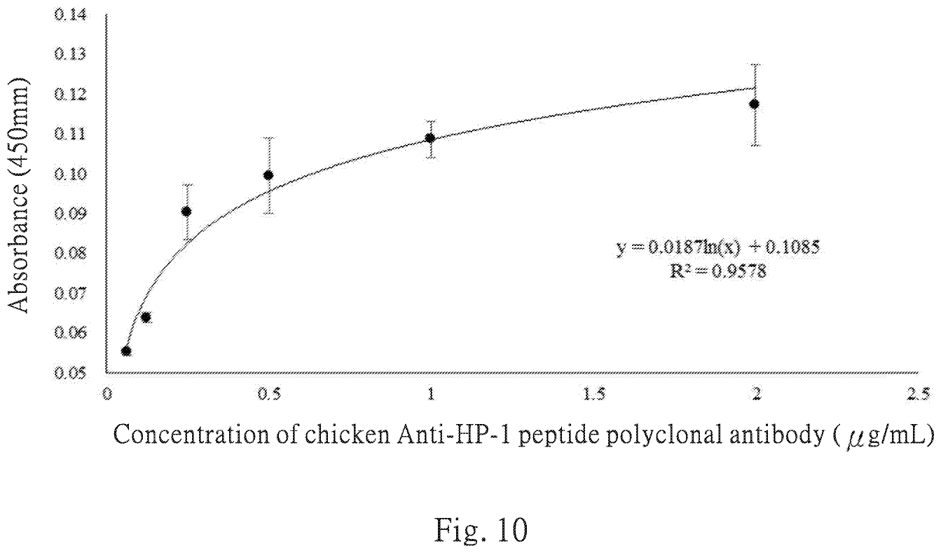

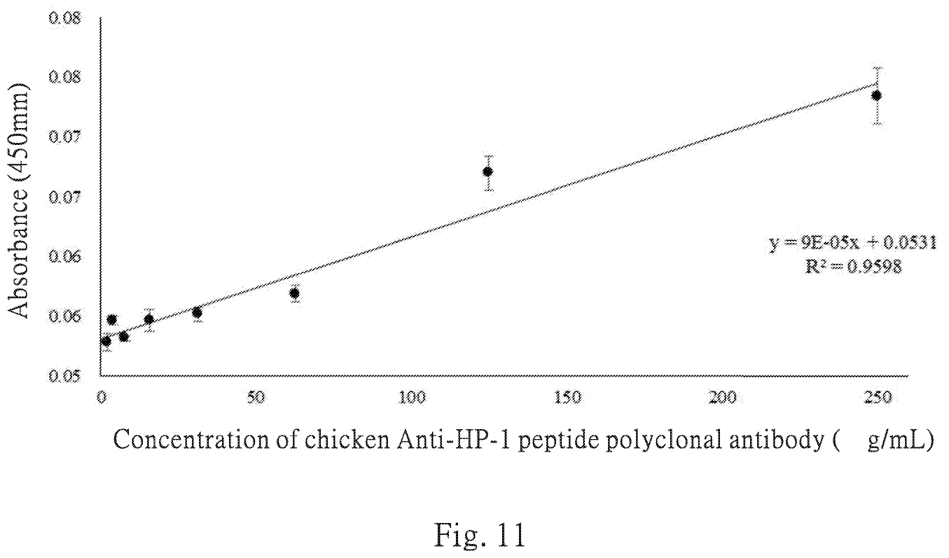

The peptide-modified assay plate obtained in Example 1 was used. The maximum antibody concentrations were 2 mg/mL and 250 μg/mL respectively, and 2-fold diluted successively. The standard curves obtained are as shown in FIGS. 10 to 11, and related data is shown in the Tables 5 and 6 below.

TABLE 5
Data determined at a maximum antibody concentration of 2 mg/mL

| | | | | | | |
|---|---|---|---|---|---|---|
| concentration | 0.0625 | 0.125 | 0.25 | 0.5 | 1 | 2 |
| (mg/mL) | | | | | | |
| Average | 0.055167 | 0.0638 | 0.090333 | 0.099433 | 0.108675 | 0.117175 |
| Absorbance | | | | | | |
| Standard | 0.000665 | 0.001203 | 0.006943 | 0.009452 | 0.004517 | 0.010186 |
| Deviation | | | | | | |

TABLE 6
Data determined at a maximum antibody concentration of 250 μg/mL

| | | | | | | | | |
|---|---|---|---|---|---|---|---|---|
| concentration | 1.953125 | 3.90625 | 7.8125 | 15.625 | 31.25 | 62.5 | 125 | 250 |
| (μg/mL) | | | | | | | | |
| Average | 0.052833 | 0.054667 | 0.053167 | 0.054633 | 0.055175 | 0.0569 | 0.066967 | 0.073433 |
| Absorbance | | | | | | | | |
| Standard | 0.0754% | 0.0377% | 0.0249% | 0.0929% | 0.0606% | 0.0714% | 0.1400% | 0.2334% |
| Deviation | | | | | | | | |

It can be known from the above results that, in an environment with low concentration of antibodies, assay with a conventional ELSA plate modified with bovine serum albumin results in a very large standard deviation. That is, a stable, reliable and sensitive standard curve cannot be provided. Compared with the conventional modification fashion, the assay plate modified by the method disclosed in the present invention can still provide a stable and sensitive standard curve for low-concentration antibodies.

1. An assay plate, comprising
a plate body, made of a polymeric material, comprising a body and at least one cavity located in the body; and
a compound A, disposed on a surface of the cavity, having a structure represented by Formula I below: 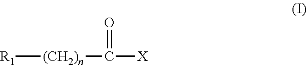
wherein:
R₁ is a hydrophobic group;
1≤n≤3; and
X is selected from the group consisting of —OH,

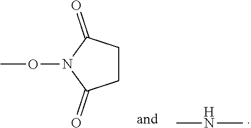

2. The assay plate according to claim 1, wherein the polymeric material is polystyrene, linked to R₁ of each of the compounds A by a hydrophobic force.

3. The assay plate according to claim 1, wherein R1 is a phenyl ring.

4. The assay plate according to claim 1, wherein the —(CH₂)ₙ— group is linear.

5. The assay plate according to claim 1, wherein the compound A is phenylacetic acid.

6. The assay plate according to claim 1, wherein the compound A is a succinate.

7. The assay plate according to claim 1, wherein the compound is an amide derivative.

8. A method for manufacturing the assay plate of claim 1, comprising: binding a hydrophobic end of at least a compound A to a plate body, wherein the compound A is selected from the group consisting of carboxylic acids, succinates and amides.

9. The method according to claim 8, comprising the following steps:
step a: contacting the compound A with the plate body to allow a moiety of the compound A to bind to a surface of the plate body; and
step b: obtaining an assay plate.

10. The method according to claim 9, wherein, in the step a, the compound A is contacted with the plate body for at least 6 hours.

11. The method according to claim 9, wherein, in the step a, the compound is contacted with the plate body by coating, perfusing or soaking.

12. The method according to claim 9, wherein, in the step a, the compound A is phenylacetic acid, and the method further comprises step a1 provided between steps a and b;
step a1: providing an activating reagent to react with the compound A bound to the plate body, and converting the compound A into a succinate compound after the reaction, wherein the activating reagent is a mixture of EDC and NHS.

13. The method according to claim 12, wherein EDC is mixed with NHS at a molar ratio of 5:1.

14. The method according to claim 12, wherein, in the step a1, the reaction time of the activating reagent with the compound A is at least 10 mins.

15. The method according to claim 12, further comprises steps a2 and a3 sequentially between the steps a1 and b, wherein:
step a2: providing a marker to react with the succinate compound obtained in the step a1, to obtain an amide compound, wherein the marker is an amine or ammonia derivative; and
step a3: masking the un-reacted succinate compound in the step a2 with ethanolamine.

16. The method according to claim 15, wherein, in the step a2, the reaction time of the marker with the succinate compound is at least 1 hour.

17. The manufacturing method according to claim 15, wherein, in the step a3, the masking reaction is continued for at least 1 hour.

18. The manufacturing method according to claim 15, wherein the reaction in the step a2 occurs in an acidic environment.


SEQUENCE LISTING (1 sequence)

SEQ ID NO: 1 - protein, 19 aa, Artificial Sequence
  Synthetic Sequence
EVYDFAFRDLCIVYRDGNP